(19) United States
(12) Patent Application Publication
(10) Pub. No.: US 2014/0003687 A1
(43) Pub. Date: Jan. 2, 2014

(54) METHOD AND SYSTEM FOR EVALUATING HEMODYNAMICS OF A BLOOD VESSEL

(71) Applicants: Liang-Der Jou, Houston, TX (US); Michel Mawad, Houston, TX (US)

(72) Inventors: Liang-Der Jou, Houston, TX (US); Michel Mawad, Houston, TX (US)

(73) Assignee: Baylor College of Medicine, Houston, TX (US)

(21) Appl. No.: 13/979,173

(22) PCT Filed: Jan. 13, 2012
(86) PCT No.: PCT/US12/21341

§ 371 Date: Sep. 11, 2013

Related U.S. Application Data
(60) Provisional application No. 61/432,730, filed on Jan. 14, 2011.

(51) Int. Cl. A61B 5/00

(52) U.S. Cl. CPC A61B 5/0033
USPC 382/130

(57) ABSTRACT

A method of angiographic evaluation of a flow diverter in a region of interest having abnormal flow in a vessel of interest, the method using digital division angiogram images at a first segment at or near a proximal end of the region of interest and at a second segment at or near a distal end of the region of interest, and observing differences in flow seen at said first and second segments.

### CROSS-REFERENCE TO RELATED APPLICATIONS

This application claims priority to U.S. Provisional Application No. 61/432,730, filed on Jan. 14, 2011, entitled Angiographic Evaluation of Flow Diverter, the disclosure of which is incorporated by reference in its entirety.

### TECHNICAL FIELD

The present disclosure generally relates to methods for evaluating hemodynamics of a blood vessel, particularly to assist in treatment of abnormal or diseased areas of blood vessels, including aneurysms and atherosclerotic disease such as plaque formation.

### BACKGROUND OF THE INVENTION

Abnormal enlargement of a cerebral artery leads to cerebral aneurysm, which is often asymptomatic until rupture. There is a high morbidity and mortality associated with hemorrhage caused by cerebral aneurysm rupture. Hemodynamics is regarded as a significant factor in the development and rupture of aneurysms, particularly cerebral aneurysm. An effective monitoring of hemodynamics of the diseased areas, such as intra-aneurysmal flow, during and after an endovascular procedure is important to the success of treating the diseased areas of a blood vessel, including an aneurysm. Coil embolization has been the preferred minimal invasive procedure for cerebral aneurysm treatment. The International Subarachnoid Aneurysm Trial (ISAT) has demonstrated that coil embolization has low mortality and morbidity rates and is a good alternative to surgical clipping.

Packing of embolic coils in an aneurysm requires a narrow neck to avoid coil herniation. As a result, not all the aneurysms can be treated by coils alone. Stent-assisted coil embolization is an option for complex aneurysms that are otherwise unsuitable for coiling. In this procedure, the stent plays an adjunct role in reconstructing parent vessel and preventing the coil from herniation. Embolization rates for both ruptured and unruptured aneurysms have improved, but complex, wide neck aneurysms remain a challenge for neuro-interventionists.

Most intracranial stents have very few struts because their only purpose is to prevent coil herniation. New generation stents (or flow diverters) have a denser mesh and lower porosity. The advantage of a flow diverter is that there may not be a need to deliver coils into the aneurysm and reconstruction of parent vessel is done with placement of a flow diverter. Multiple stents or flow diverters at the aneurysm neck can lower the porosity and reduce intra-aneurysmal flow. However, placement of multiple stents or coils across the neck may not always be feasible, and positioning one stent or coil over another to achieve the best hemodynamic effect is desired but often not possible. As such, monitoring the condition of the aneurysm during or immediately after treatment can allow precaution steps to be taken to prevent the treated aneurysm from delayed hemorrhage.

Various methods have been used to analyze hemodynamics of a portion of blood vessels, particularly cerebral aneurysms and coil or stent performance during and after treatment. Conventional methods often rely on contrast transport pattern on two dimensional (2D) digital subtraction angiography (DSA). These methods, however, suffer from various disadvantages. For instance, parent vessels or small branches laying on top of an aneurysm can prohibit the use of a region of interest (ROI) around the aneurysm. Many conventional approaches require an unobstructed view of the aneurysm under investigation. Considering that most aneurysms are surrounded by branching vessels, this requirement prohibits many clinical applications. In addition, how much convection (or flow) reduction is sufficient to ensure a complete embolization is unclear from these methods. Further, conventional approaches are likely not suitable to evaluate intra-aneurysmal flow after coil embolization since the aneurysm is no longer visible on the digital subtraction angiography (DSA) images. This presents a challenge to neuro-interventionalists during the treatment because a decision needs to be made on whether additional treatment, e.g., flow diverters or coils, are required for complete embolization. In addition, these conventional analytical tools often require lengthy calculation and offer no immediate hemodynamic assessment. These conventional approaches are also constrained by various assumptions that are regarded as limitations of numerical models. Moreover, conventional methods often require a lengthy computation times and a large amount of data in a cardiac cycle.

### SUMMARY OF THE INVENTION

According to one aspect of the present disclosure, there is provided a method for evaluating hemodynamics of a region of interest, said method comprising the steps of: injecting a contrast agent upstream of a region of interest of a blood vessel; creating a plurality of 3D digital subtraction images and a plurality of 2D images of said blood vessel based at least on a measured signal of said injected contrast agent; creating a plurality of digital division angiogram images from said 2D images; selecting a first segment in said vessel at or near the proximal end of said region of interest, said first segment having a geometry characterized by having at least one axis being orthogonal to the centerline of the blood vessel; measuring mean signal intensity for the first segment from said digital division angiogram images; mapping said plurality of digital division angiogram images back to said 3D images to obtain a concentration profile of said first segment.

In one embodiment, the method further comprises the steps of: selecting a second segment in said blood vessel at or near the distal end of said region of interest, said second segment having a geometry characterized by having at least one axis being orthogonal to the centerline of the blood vessel; measuring mean signal intensity for the second segment from said digital division angiogram images; and mapping said plurality of digital division angiogram images back to said 3D images to obtain a concentration profile of said second segment. In another embodiment, the method further comprises the step of comparing the concentration profile of said first segment with the concentration profile of said second segment to determine the hemodynamics of the region of interest.

In one embodiment, the contrast agent is injected prior to treatment of said region of interest, wherein said treatment is configured to alter the hemodynamics of said region of interest. In another embodiment, the method further comprises the steps of injecting a contrast agent upstream of a region of interest of a blood vessel after said treatment; creating a plurality of 3D digital subtraction images and 2D images of said blood vessel based on said injected contrast agent; creating a plurality of digital division angiogram images from said 2D images; measuring mean signal intensity for the first segment from said digital division angiogram images; mapping said plurality of digital division angiogram images back to said 3D images to obtain a concentration profile of said first segment after said treatment; measuring mean signal intensity for the second segment from said digital division angiogram images; mapping said plurality of digital division angiogram images back to said 3D images to obtain a concentration profile of said second segment after said treatment; and comparing the concentration profiles of the first and second segments obtained prior to treatment with the concentration profiles of the first and second segments obtained after said treatment to evaluate the performance of said treatment.

In one embodiment, the method further comprises the steps of injecting a contrast agent upstream of a region of interest of a blood vessel during said treatment; creating a plurality of 3D digital subtraction images and 2D images of said blood vessel based on said injected contrast agent; creating a plurality of digital division angiogram images from said 2D images; measuring mean signal intensity for the first segment from said digital division angiogram images; mapping said plurality of digital division angiogram images back to said 3D images to obtain a concentration profile of said first segment after said treatment; measuring mean signal intensity for the second segment from said digital division angiogram images; mapping said plurality of digital division angiogram images back to said 3D images to obtain a concentration profile of said second segment after said treatment; and comparing the concentration profiles of the first and second segments obtained prior to treatment with the concentration profiles of the first and second segments obtained during said treatment to determine whether additional treatment is needed.

In one embodiment, the treatment comprises use of flow diverter. In another embodiment, the treatment comprises coil embolization. In one embodiment, the region of interest is an aneurysm. In another embodiment, the region of interest is the site of atherosclerotic disease.

In one embodiment, the 3D digital subtraction angiogram images and said 2D images are X-ray images. In another embodiment, the 2D images are obtained at a frame rate of 30 f/s or greater. In another embodiment, the first segment and said second segment are substantially equidistant from said region of interest. In another embodiment, the contrast agent comprises (1-N,3-N-bis(2,3-dihydroxypropyl)-5-[N-(2,3-dihydroxypropyl)acetamido]-2,4,6-triiodobenzene-1,3dicarboxamide). In another embodiment, the step of injecting a contrast agent comprises injecting a contrast agent at an injection rate of between about 1 mL/s and 3 mL/s. In yet another embodiment, the step of injecting a contrast agent comprises injecting a contrast agent at an injection rate of about 2 mL/s or lower.

According to another aspect of the present disclosure, there is provided a method for evaluating hemodynamics of a region of interest, said method comprising the steps of: injecting a contrast agent upstream of a region of interest of a blood vessel;

creating a plurality of 3D digital division angiogram images and a plurality of 2D digital division angiogram images of said blood vessel based at least on a measured signal of said injected contrast agent; selecting a first segment in said vessel at or near the proximal end of said region of interest, said first segment having a geometry characterized by having at least one axis being orthogonal to the centerline of the blood vessel; determining the volume of said first segment, said determining comprises projecting said plurality of said 2D digital subtraction angiogram images back to said 3D digital subtraction angiogram images; obtaining a concentration profile for said first segment using said determined volume to normalize the total signal of the injected contrast agent at said first segment.

In one embodiment, the method further comprises the steps of: selecting a second segment in said vessel at or near the proximal end of said region of interest, said first segment having a geometry characterized by having at least one axis being orthogonal to the centerline of the blood vessel; determining the volume of said second segment, said determining comprises projecting said plurality of said 2D digital subtraction angiogram images of said second segment back to said 3D digital subtraction angiogram images; and obtaining a concentration profile for said second segment using said determined volume to normalize the total signal of the injected contrast agent at said second segment. In another embodiment, the method further comprises the step of comparing the concentration profile of said first segment with the concentration profile of said second segment to determine the hemodynamics of the region of interest. In yet another embodiment, the comparing step comprises determining a flow rate in the region of interest based at least on the difference between the concentration profile of the first segment and the concentration profile of the second segment.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 shows the signal for increasing concentrations of contrast media;

FIG. 2 demonstrates the information provided by the 3D images and how this relates to the 2D projection;

FIG. 3 is a plot showing contrast concentration in a vessel as demonstrated by signal for systole and diastole periods;

FIG. 4A illustrates proximal and distal locations to an area of interest of a blood vessel that has normal flow, e.g., no aneurysm or other condition of abnormal flow;

FIG. 4B shows concentration profiles demonstrating hemodynamics before and after compensation of time delay for flow at the proximal and distal locations to an area of interest of the blood vessel in FIG. 4A according to the aspects of the present disclosure;

FIG. 5A illustrates proximal and distal locations to an area of interest of a blood vessel that has abnormal flow condition, e.g., an aneurysm;

FIG. 5B shows concentration profiles demonstrating hemodynamics before and after compensation of time delay for flow at the proximal and distal locations to an area of interest of the blood vessel in FIG. 5A according to the aspects of the present disclosure;

FIGS. 6A and 6B show exemplary use of concentration profiles demonstrating hemodynamics to evaluate the performance of treatment of a diseased area of a blood vessel according to the aspects of the present disclosure;

FIG. 7A provides an illustration of time slices which are related to contrast agent concentration;

FIG. 7B shows a concentration profile of the corresponding signals of the concentration of FIG. 7A;

FIGS. 8A and 8B show exemplary concentration profiles demonstrating hemodynamics of a diseased area of a blood vessel before and after treatment of that diseased area according to the aspects of the present disclosure;

FIGS. 9A-9C show exemplary use of concentration profiles demonstrating hemodynamics of a diseased area of a blood vessel before treatment, during treatment, and 24 hours later, respectively, according to the aspects of the present disclosure;

FIGS. 9D-9E show the portion of the concentration profiles of FIGS. 9A and 9C between 4-6 seconds, respectively;

FIG. 10A illustrates a vessel with an aneurysm;

FIG. 10B shows exemplary use of concentration profiles to evaluate hemodynamics at the region of interest, e.g., the aneurysm of FIG. 10A, according to the aspects of the present disclosure;

FIG. 11A is a photograph of a blood vessel with an aneurysm having some vessel overlap prior to treatment;

FIG. 11B-D show exemplary use of concentration profiles demonstrating hemodynamics of the aneurysm of FIG. 11A before treatment, immediately after treatment, and 24 hours later to evaluate performance of the treatment according to the aspects of the present disclosure;

FIG. 12A is a photograph of a blood vessel with four separate aneurysms prior to treatment;

FIG. 12B-D show exemplary use of concentration profiles demonstrating hemodynamics of the aneurysm of FIG. 12A before treatment, immediately after treatment, and 24 hours later to evaluate performance of the treatment according to the aspects of the present disclosure;

FIG. 13A is a photograph of a blood vessel with two separate aneurysms prior to treatment;

FIG. 13B-D show exemplary use of concentration profiles demonstrating hemodynamics of the aneurysm of FIG. 13A before treatment, immediately after treatment, and 24 hours later to evaluate performance of the treatment according to the aspects of the present disclosure;

FIG. 14A shows the concentration profiles in FIGS. 13B-D of the proximal region of interest before treatment, immediately after treatment, and 24 hours later in the same graph;

FIG. 14B shows the concentration profiles in FIGS. 13B-D of the distal region of interest before treatment, immediately after treatment, and 24 hours later in the same graph;

FIG. 15 shows concentration profiles demonstrating hemodynamics of the aneurysm of FIG. 13A two weeks after treatment;

FIG. 16A is a photograph of the aneurysm of FIG. 13 taken 2 weeks after treatment;

FIGS. 16B-D show the portion of the concentration profiles of FIGS. 13B, 13D and 15 between 4-6 seconds, respectively;

FIG. 17 illustrates one exemplary way according to the aspects of the present disclosure to quantify the transport of contrast agent transport at the proximal portion and the distal portion to the region of interest, as well as the region of interest;

FIG. 18 illustrates concentration profiles of three regions of interest selected within about an area of interest of 4 cm demonstrating similar hemodynamics of these regions according to the aspects of the present disclosure;

FIG. 19A shows concentration profiles demonstrating hemodynamics at the proximal portion, the distal portion, and at the region of interest, where the ratio of the flow rate at the aneurysm to the flow rate at the proximal portion is high according to the aspects of the present disclosure;

FIG. 19B shows concentration profiles demonstrating hemodynamics at the proximal portion, the distal portion, and at the region of interest, where the ratio of the flow rate at the aneurysm to the flow rate at the proximal portion is lower than the ratio of FIG. 19A according to the aspects of the present disclosure

FIG. 19C shows concentration profiles demonstrating hemodynamics at the proximal portion, the distal portion, and at the region of interest, where the ratio of the flow rate at the aneurysm to the flow rate at the proximal portion is lower than the ratio of FIG. 19B according to the aspects of the present disclosure;

FIG. 20A shows exemplary use of concentration profiles demonstrating hemodynamics of the regions around an aneurysm after three flow diverters have been placed in the aneurysm to evaluate the effect of the flow diverters according to the aspects of the present disclosure; and

FIG. 20B shows exemplary use of concentration profiles demonstrating hemodynamics of the regions around an aneurysm after coil embolization to evaluate the effect of the treatment according to the aspects of the present disclosure.

### DETAILED DESCRIPTION OF THE EMBODIMENTS

As used herein, “a” or “an” means one or more. Unless otherwise indicated or clear from the context, the singular contains the plural and the plural contains the singular. For example, a reference to “images” should be understood to mean “one or more images”. Similarly, a reference to an “image” should be understood to mean “one or more images”.

The various embodiments of the present disclosure provide effective evaluation of hemodynamics, such as intra-aneurysmal flow, based on cerebral angiography. The embodiments of the present disclosure allow for evaluation of the condition of cerebral aneurysm during or immediately after treatment so precaution can be taken to prevent an aneurysm from delayed hemorrhage.

The embodiments of the present disclosure offer several advantages over traditional techniques. For instance, embodiments of the present disclosure provide an absolute scale of the attenuation that is proportional to the contrast concentration. This feature allows a comparison of images acquired on different patients at different times. Further, embodiments of the present disclosure provide a beat-to-beat variation of ultra-aneurysmal flow which is often ignored in traditional analyses. The beat-to-beat variation is particularly important when flow diverters or coils are evaluated according to the aspects of the present disclosure. Also, the embodiments of the present disclosure are less affected by an overlap of an aneurysm or diseased area by a parent vessel or other vessels. It is likely that other section of parent vessel will be available for measurement. Certain embodiments of the present disclosure use the cross-section area and diameter of a parent vessel of an aneurysm, which is easier to do than a similar measurement on the aneurysm itself.

Additional advantages provided by the embodiments of the present disclosure include the ability to examine activities taking place within the aneurysm by comparing signals proximal to the aneurysm with signals distal to the aneurysm. The difference between these signals is preferably independent of imaging processing. Further, a lower dosage of contrast agent can be used in the embodiments of the present disclosure, thereby potentially lowering the total contrast agent injected into a patient in the procedure. In addition, a low-dose injection prevents signals from saturation and allows an observation of dynamic behavior of contrast transport, for which the attenuation and concentration are linear. Also, the embodiments of the present disclosure can serve as a real-time evaluation of the conditions of the diseased area, such as an aneurysm. The embodiments of the present disclosure can be implemented in a clinical environment and provide an easy and quick evaluation of flow diversion efficiency.

Moreover, the embodiments of the present disclosure provide a more accurate diagnostic measure for the performance of a treatment, such as a flow diverter or coil, for treating conditions of abnormal flow rates in blood vessels, for example, such as those caused by the presence of an aneurysm or atherosclerotic disease. For instance, the embodiments of the present disclosure allow for more precise determination of the amount of flow modification that is necessary to eliminate the condition of abnormal flow rate.

According to one aspect of the present disclosure, evaluation of hemodynamics by creating digital division angiogram (DDA) images from digital subtraction angiogram (DSA) images. DSA images are usually acquired by exposing an area of interest, e.g. a diseased area of a blood vessel such as an aneurysm, with time-controlled X-rays while injecting contrast medium into the blood vessels. The images obtained often include all overlying structure besides the area of interest. One way of isolating the structures of interest is to acquire a mask image, which is subtracted from the images acquired with the contrast agent to generate the DSA images. The mask image is generally an image of the same area before the contrast is administered. The radiological equipment used to capture images of the blood vessel is usually an image intensifier, which will keep producing images of the same area at a set rate (1-6 frames per second), taking all subsequent images away from the original “mask” image to generate DSA images. The concentration rate of the contrast medium being injected and duration of injection can be controlled.

Injection of contrast medium provides the basis for clinical diagnosis, and the wash-in and wash-out of contrast medium offers a convenient way to observe the contrast dynamic behavior. The time course of the signal at a sight of interest, e.g., an aneurysm, is often fitted into a mathematical formula to determine contribution from various flow components (convention, diffusion, or mean transit time).

In the preferred embodiment, the DSA images are obtained during a controlled injection of contrast agent or agent at a low injection speed. With a controlled injection of contrast agent at low speed, the generated images have signal intensity that is proportional to the attenuation or contrast concentration. One way to obtain DDA images is instead of subtracting the masked image, each pixel in an image is divided by the pixel at the same location in the masked image.

In one embodiment, the dimensions of the parent vessel are preferably determined on three dimensional (3D) images and temporal variations of signal intensity at two sections of the parent vessel at opposite sides of the aneurysm are measured. Preferably, these signal intensities are resealed by parent artery sizes to give an absolute measure of contrast concentration. In certain embodiments, the measure of contrast concentration of the two sections can be presented in either percentage of contrast agent in the blood or Hounsfield unit.

In other embodiments, temporal changes of contrast concentration reflect the pulsatility of blood flow and intra-aneurysmal flow, and the difference between signals proximal and distal to an aneurysm represents the level of intra-aneurysmal flow ratio. The flow ratio preferably indicates the amount of blood flow entering an aneurysm.

According to another aspect of the present disclosure, dynamic changes of contrast concentration reflect the pulsatility of blood flow and intra-aneurysmal flow, and the difference between signals proximal and distal to an aneurysm represents the level of intra-aneurysmal flow and therapeutic effect of a treatment, such as flow diverters or coils. In one embodiment, the mean concentration of contrast medium can be derived from these digital division angiographic images. Variation of this mean concentration may indicate the pulsatility of blood flow and intra-aneurysmal flow. The difference between contrast concentrations proximal and distal (with regard to the origin of flow) to the location of the cause of abnormal flow (for example, an aneurysm) represents the level of the perturbation of normal flow (in the case of an aneurysm, this is a measure of intra-aneurysmal flow). In the preferred embodiment, the reduction of this difference in proximal and distal concentration contrast indicates the decrease in perturbation to normal flow, and thus, the effectiveness of the treatment. Accordingly, the disappearance or substantial disappearance of this difference in proximal and distal concentration contrast indicates when treatment of the abnormal flow is successful.

The following examples provide exemplary or preferred embodiments of the present disclosure, which are only for illustrative purposes and not intended to limit the scope of the present disclosure.

### Example 1

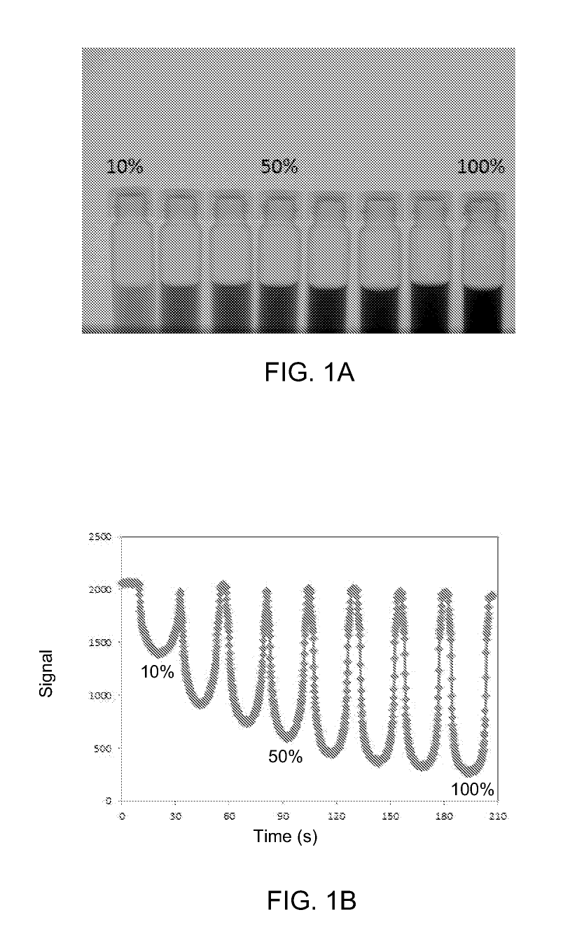

In one embodiment, dosage of the contrast agent (e.g., concentration and speed of injection) is determined prior to acquiring angiographic images. Referring to FIG. 1A, preferably, different ratios of contrast media are measured. In particular, eight bottles of contrast media were mixed with various amount of saline so the ratio of the contrast component in each solution varies from 10% to 100%. These bottles were imaged by X-ray and their signal intensities were measured. Signal from solution with 10% and 50% contrast medium reached 37% and 82% of the signal from pure contrast medium (100%), respectively. Accordingly, the first 50% contrast media provides a greater contrast ratio, and the gain from an additional 50% is merely 18% more signal and less significant. The digital subtraction technique does not produce a linear relationship between the signal and concentration of contrast medium.

In one embodiment, to fully appreciate the signal change during contrast injection, the contrast concentration needs to be limited to no more than 50% so the relationship between the contrast concentration and X-ray signal is approximately linear. Since contrast medium is diluted by the blood immediately after injection, the highest contrast concentration will occur at the diastolic period when the blood flow rate is the lowest. If the contrast medium is injected at half of the mean flow rate for a given vessel, then the maximum concentration in the vessel will be no more than 50% and the signal change due to injection will be visible throughout the period. In one embodiment, such as for an internal carotid artery, an injection rate of preferably between about 1 mL/s and 3 mL/s (milliliters per second), and more preferably about 2 mL/s, is sufficient for observation of dynamic change. In other embodiments, the injection rate can be manipulated to be less than 2 mL/s or greater than 2 mL/s on a case-by-case basis.

FIG. 1B shows the signal for increasing concentrations of contrast media. As shown, as the concentration increases, the X-ray density of the solution increases, thereby decreasing transmission of X-ray radiation. The baseline (shown at an approximate y-axis value of 2000) is the signal level with no contrast agent (in this case, X-ray dye), giving a 100% transmission or near 100% transmission of X-ray radiation.

Although any suitable contrast agent may be used, the present studies used iohexyl (1-N,3-N-bis(2,3-dihydroxypropyl)-5-[N-(2,3-dihydroxypropyl)acetamido]-2,4,6-triiodobenzene-1,3-dicarboxamide), commercially sold as Omnipaque. In the preferred embodiment, the measured signal was an X-ray signal. X-ray radiation was impinged on the region of interest and the attenuation of the X-ray was the basis of the signal. The contrast agent increases the density of the material under study with regard to transmission of X-ray radiation. An increase in the attenuation in a given region is indicative of the presence of more contrast agent in that region. It is therefore desirable to work with concentrations of contrast agent that result in linearity of response for signal intensity versus concentration of contrast agent. Although the examples and the discussion herein use iohexyl as contrast agent and X-ray radiation as the interrogation beam, it should be understood that other regions of the electromagnetic spectrum may be used and other contrast agents may also be used, so long as the measured signal can accurately be used as measure of the amount of contrast agent in the region being interrogated.

While most of the remainder of the discussion focuses on the application of the method of the present invention to the diagnosis of performance of flow diverters as applied to aneurysms, it should be understood that the embodiments of the present disclosure are not so limited and are applicable to evaluate hemodynamics of a blood vessel, particularly for treatment purposes such as the diagnosis of performance of various treatments, e.g., flow diverters or coils, as applied to conditions of abnormal vessel flow, such as, but not limited to atherosclerotic disease such as plaque formation.

In one embodiment, to determine the concentration of contrast agent, the X-ray signal at each pixel is a function of the attenuation coefficient (μ) and penetration depth (x), and it can be written as:

Iₓ=I₀e^{-μx}

The attenuation coefficient is proportional to the contrast medium concentration, or μ=kc, where k is a constant and c is the concentration of contrast medium. For this reason, images are preferably not digitally subtracted by the masked image. Instead, each pixel in an image is divided by the pixel at the same location in the masked image, and the logarithm of the result will be proportional to the product of the attenuation coefficient (μ) and penetration depth (x). After this digital division and logarithmic process, the value at each pixel is proportional to μx.

The sum of all the pixels in a given region of interest (A_{yz}) is:

S=∫_{z}∫_{y}μ·xdydz

This can be rewritten as:

S=∫_{z}∫_{y}μ·x·dydz=∫_{z}∫_{y}∫ₓμ·dxdydz.

Therefore, this sum is a volume integral of the attenuation coefficient. This sum S is preferably obtained from 2D X-ray images. In one embodiment, because μ=k·c the mean concentration of contrast medium (Cₘₑₐₙ) is related by the sum of all pixels in a given region A_{yz}. Cₘₑₐₙ can be expressed as follows:

Cₘₑₐₙ=∫_{z}∫_{y}∫ₓc·dxdydz/∫_{z}∫_{y}∫ₓdxdydz=∫_{z}∫_{y}∫ₓc·dxdydz/V_{xyz}=S/K·V_{xyz}).

Evaluation of S actually does not involve a volume integral of μ. From two-dimensional (2D) digital division angiographic images, S=s·w·d=k Cₘₑₐₙ·V_{xyz} where s is the mean signal intensity in the region of interest. Since the volume of the region of interest is V_{xyz}=A·d, the mean concentration is (s·w·d)/(k·A·d)=(s·w)/(k·A), in which A is the area of the cross-section, w is the width and d is the diameter, and s is the mean signal intensity of the digital division images in the region of interest. This formula greatly simplifies the determination of contrast concentration. Even though d does not appear in the calculation, a finite size d will reduce noises by averaging over a finite region. Therefore, in the preferred embodiment, the mean concentration at any given cross-section in a vessel can be determined.

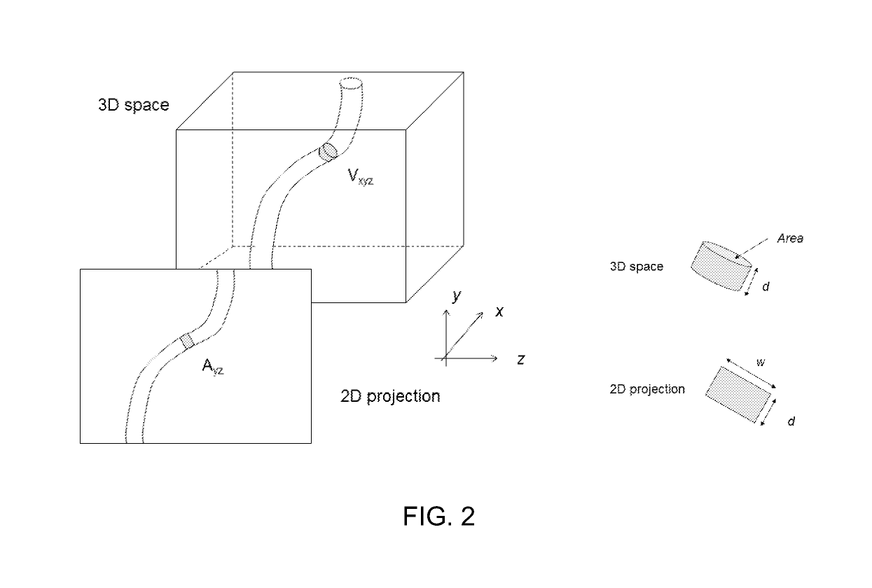

FIG. 2 demonstrates the information provided by the 3D images and how this relates to the 2D projection. In the preferred embodiment, the 3D data acquisition using significant signal averaging over the volume for better data. The concentration in a vessel is then preferably determined. Given a specific cross-section in a vessel, the total signal S can be measured easily on the 2D X-ray images and V_{xyz} can be measured on three-dimension (3D) images. Then the concentration of contrast medium is proportional to S/V_{xyz}. While it does not represent the real concentration, it differs from the real concentration by a constant k that depends on X-ray voltage.

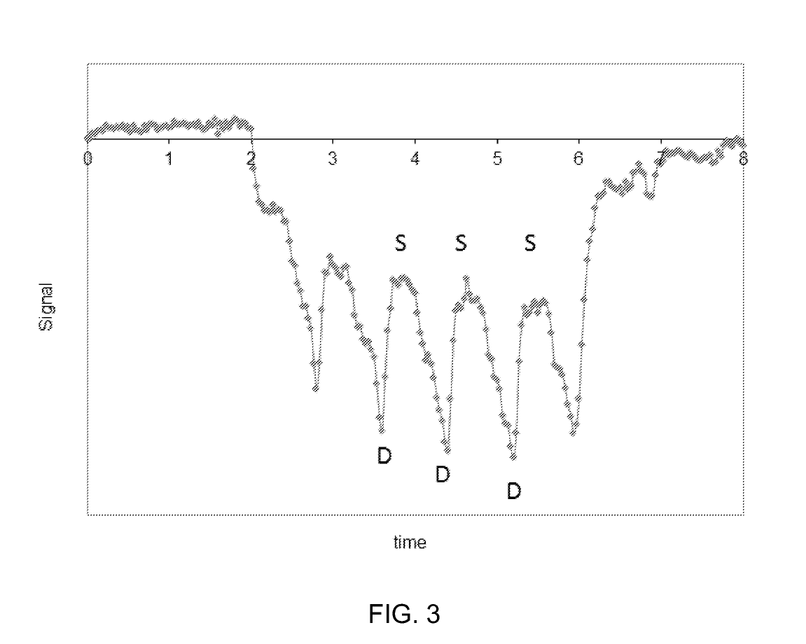

FIG. 3 is a plot showing contrast concentration in a vessel as demonstrated by signal for systole periods, indicated by the letter “S,” and diastole periods, indicated by the letter “D” in the y-axis with respect to time in the x-axis. As shown by FIG. 3, at low injection rate, there will be significant variation in signal in the vessel. However, the signal variation indicates the duration of each period for both systole (S) and diastole (D). At peak systole, the blood flow rate is the highest and contrast medium is diluted the most so the signal is lower (e.g., less negative, or closer to 0, as shown in FIG. 3). At diastole, the blood flow rate is lower and contrast medium is less diluted so the signal is higher (more negative in the figure because of the negative sign in the X-ray attenuation formula).

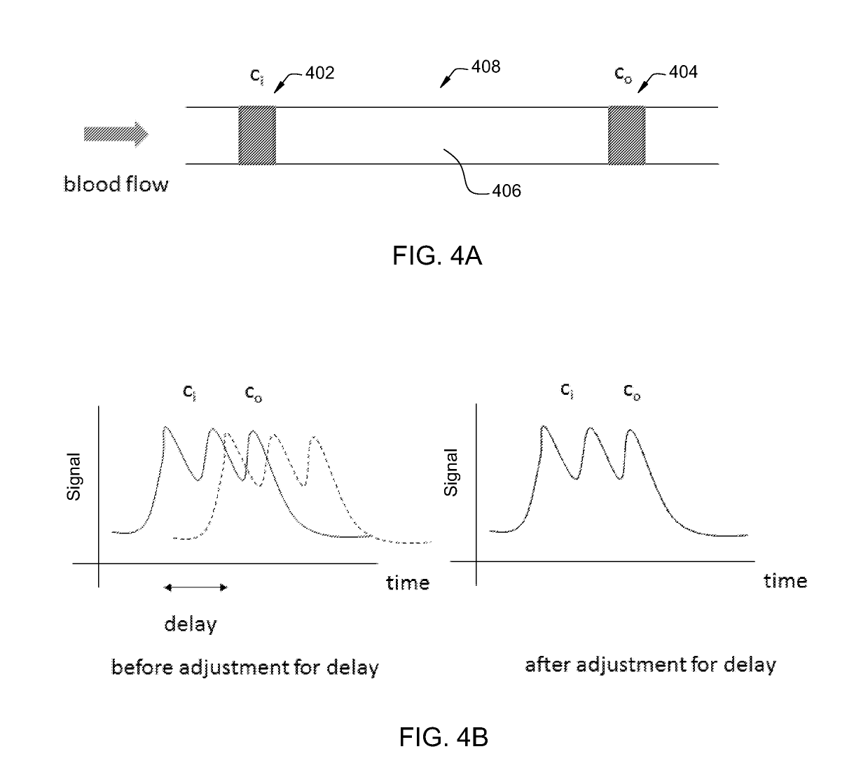

FIG. 4A illustrates proximal location 402 and distal location 404 to area of interest 406 of blood vessel 408 that has normal flow, e.g., no aneurysm or other condition of abnormal flow. FIG. 4B shows compensation of time delay for flow at proximal location 402 and distal location 404 to area of interest 406 of blood vessel 408 in FIG. 4A. Referring to FIG. 4A, for an aneurysm with normal flow, any two sections is the same. That is, the concentration of contrast medium at proximal location 402, designated as cᵢ, is the same as the concentration of contrast medium at distal location 404, designated as cᵢ. In other words, cᵢ=cₒ.

Referring to FIG. 4B, cₒ differs from cᵢ by a time delay, which depends on the distance between proximal location 402 and distal location 404. In the preferred embodiment, this time delay is used to calculate the flow rate in vessel 408. The plot for the concentration of contrast medium at proximal location 402, designated as cᵢ with respect to time is represented by the solid line in FIG. 4B. The plot for the concentration of contrast medium at distal location 404, cₒ, with respect to time is demonstrated by the dashed line in FIG. 4B. Referring to FIG. 4B, after adjustment for this time delay, cᵢ of proximal location 402 is often very close to cₒ of distal location 404 for vessel 408, which exhibits normal flow. In some instances, the effects diffusion that occurs in the region between point i and point o can contribute to a difference between cᵢ and cₒ; however, such effects should be minor.

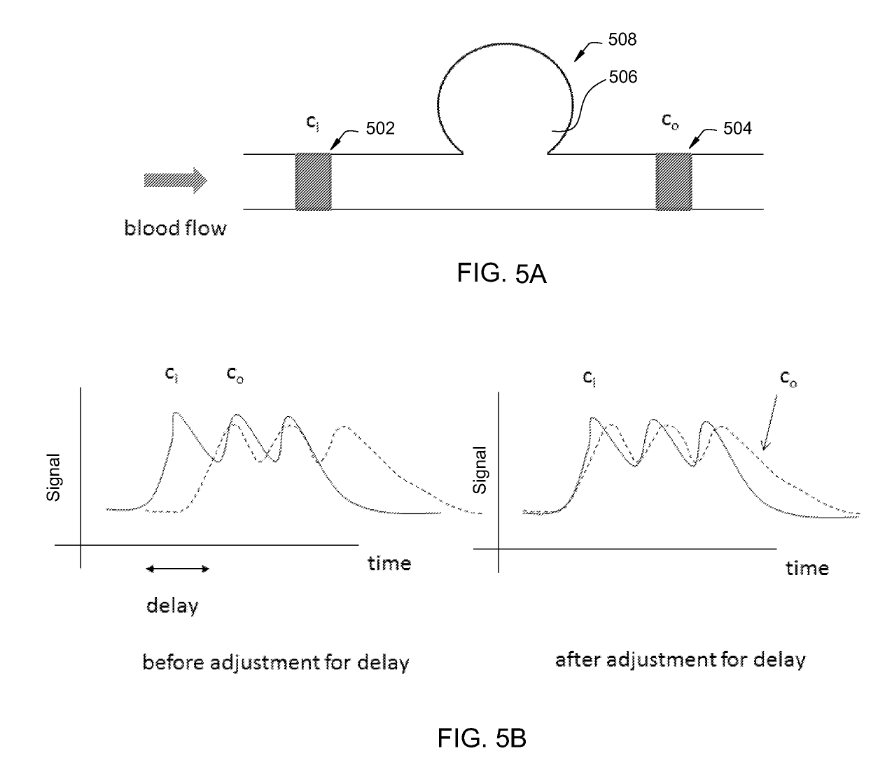

FIG. 5A illustrates proximal location 502 and distal location 504 to area of interest 506, i.e., aneurysm 506, of blood vessel 508 that suffers from abnormal flow. FIG. 5B shows compensation of time delay for flow at proximal location 502 and distal location 504 to aneurysm 506 of blood vessel 508 in FIG. 5A. In one embodiment, the effect of intra-aneurysmal flow for vessel 508 can be demonstrated by the difference between cᵢ and cₒ. For instance, when an aneurysm is present, cᵢ will be different from cₒ after adjustment for the time delay. The plot for the concentration of contrast medium at proximal location 502, cᵢ, with respect to time is represented by the solid line in FIG. 5B. The plot for the concentration of contrast medium at distal location 504, cₒ, with respect to time is demonstrated by the dashed line in FIG. 5B. The difference between cᵢ and cₒ is caused by the flux of contrast medium into and out of the aneurysm and mixing of blood with contrast medium in the aneurysm. After the adjustment for time delay and the two plots of cᵢ and cₒ are superimposed, it can be seen that during systole, cᵢ is higher than cₒ, meaning that some contrast medium is traveling into the aneurysm. During diastole, cᵢ is lower than cₒ so contrast medium is leaving the aneurysm. During the period of injection, part of contrast medium will accumulate within the aneurysm. However, accumulated contrast medium may leave as soon as the injection ends, and a prolonged period of contrast washout can be observed where cₒ>cᵢ at the end of imaging process.

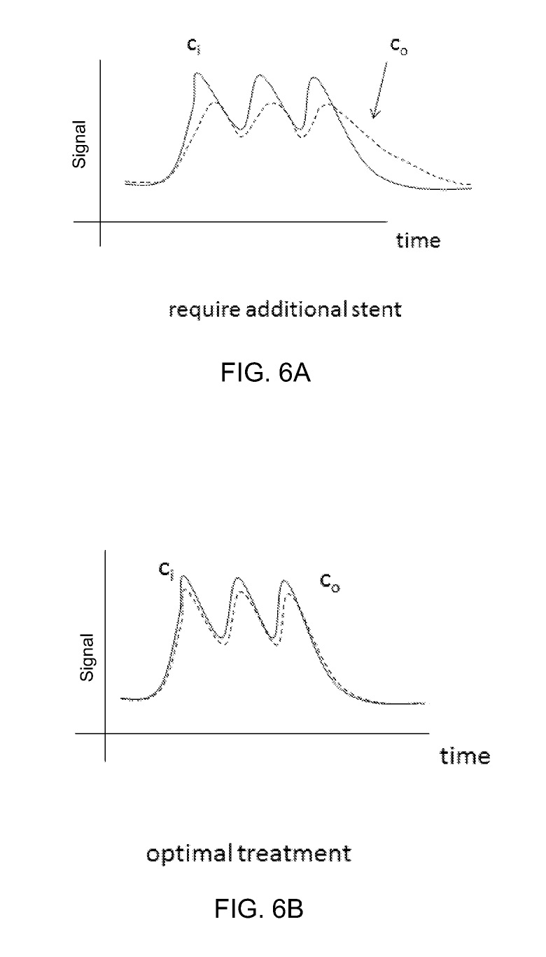

According to another aspect, the embodiments of the present disclosure can be used to evaluate the effectiveness of a treatment, such as placement of flow diverters or coils at appropriate locations to reduce the perturbations to normal flow. The purpose of a particular treatment, such as flow diverter or coil, is to reconstruct parent vessel, e.g., by reducing or eliminating blood flow to the aneurysm from the circulatory system. Suppose that a flow diverter has eliminated the intra-aneurysmal flow, the parent vessel is reconstructed to normal condition or close to normal condition, and the aneurysm no longer poses a significant risk. In the preferred embodiment, cᵢ=cₒ, or cᵢ closely matches cₒ, thereby similarly resembling an injection of contrast medium into a normal vessel, such as that shown in FIGS. 4A and 4B. Accordingly, the difference between cᵢ and cₒ is preferably used to evaluate the effectiveness of a treatment. This is demonstrated in FIGS. 6A and 6B. FIG. 6B demonstrates the difference between cᵢ and cₒ already adjusted for the time delay. In FIG. 6A, a plot for the concentration of contrast medium at proximal location 502, cᵢ, with respect to time is represented by the solid line. In FIG. 6A, a plot for the concentration of contrast medium at distal location 504, cₒ, with respect to time is represented by the dashed line. The time adjusted graphs of cᵢ and cₒ do not closely match or resemble one another, indicating a sub-optimal treatment or additional treatment is needed, e.g., more flow diverters or coils, to return blood flow to normal or close to normal conditions. The time adjusted graphs cᵢ and cₒ in FIG. 6A also resembles the time adjusted graphs cᵢ and cₒ of blood vessel 508 of FIG. 5B, which exhibits abnormal flow.

FIG. 6B demonstrates the difference between cᵢ and cₒ already adjusted for the time delay. The time adjusted graphs cᵢ and cₒ in FIG. 6B also resembles the time adjusted graphs cᵢ and cₒ of blood vessel 408 of FIG. 4B, which has normal flow. In FIG. 6B, a plot for the concentration of contrast medium at proximal location 502, cᵢ, with respect to time is represented by the solid line. In FIG. 6B, a plot for the concentration of contrast medium at distal location 504, cₒ, with respect to time is represented by the dashed line. The time adjusted graphs of cᵢ and cₒ closely match or resemble one another, indicating an effective treatment and return of blood flow to normal or close to normal conditions.

An example of implementation of the embodiments according to the aspects of the present disclosure is now provided. In the preferred embodiment, a 3D digital subtraction images and 2D X-ray images are acquired at a high frame rate of more than 15 frames/second (f/s), and preferably 30 frames/second (f/s) or more, with a slow injection, e.g., about 2 mL/s as discussed above. The series of 2D X-ray images will first be divided by the first frame (i.e., the mask), which is the frame before contrast medium arrives, and then the logarithm of the division is taken to generate a new series of images (digital division angiographic images or DDA). The pixels outside the lumen preferably have values close to zero, and the pixels inside the lumen preferably have negative values. In the preferred embodiment, the values of pixels inside the lumen are proportional to the attenuation coefficient of contrast medium.

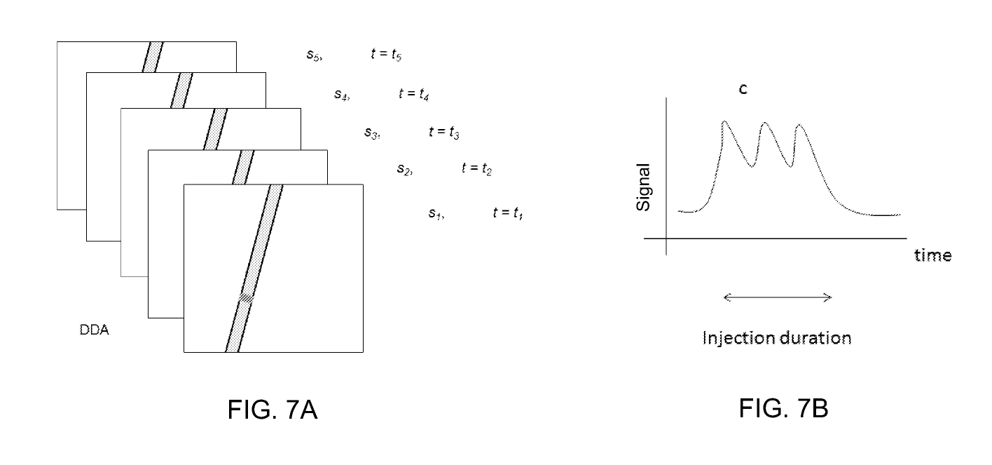

In the preferred embodiment, a section of the vessel proximal to an area of interest, e.g., an aneurysm, is first selected. This section is preferably perpendicular (i.e., orthogonal) to the centerline of the vessel. In one embodiment, a thickness of the selected section is chosen to help reduce noises in the images. After the section is selected, the mean DDA signal of that section (s in early derivation) is calculated for each frame. The width of this section needs also to be measured from the images, which is w. The section in 2D DDA is mapped back to the 3D images, and the area of the section of vessel in 3D images is A. The concentration profile at a given section of vessel then is (s·w)/(k·A) from the earlier derivation provided above. In the preferred embodiment, the same voltage is used throughout the X-ray acquisition; thus, the constant k can be ignored. FIG. 7 provides an illustration of time slices and the corresponding signals, which are related to contrast agent concentration as described earlier.

In the preferred embodiment, a section of vessel distal to the area of interest, e.g., the aneurysm, is similarly selected. The procedure of determining contrast profile for the distal section is repeated. These two contrast profiles (e.g., cᵢ and cₒ, as described for FIGS. 4A, 4B, 5A, 5B, 6A, and 6B) represent the concentration variation at the proximal location and distal location to the area of interest in the vessel. In the preferred embodiment, these two profiles (cᵢ and cₒ) are obtained from the same series of images; thus, the constant k remains same. Accordingly, in the preferred embodiment, these profiles can be compared directly without the constant k.

The time delay (Δt) between cᵢ and cₒ preferably is determined from the temporal difference of peaks between cᵢ and cₒ. Manipulation of the time delay preferably allows for estimation of the flow rate in the vessel. For example, the time delay can be measured from the distance between peaks of c, and cₒ. The time axis for the cₒ can be readjusted by this time delay (Δt) so that both cᵢ and cₒ peaks at the same time. This adjustment compensates for the effect of contrast propagation in the vessel. As discussed above, for a normal vessel, these two profiles cᵢ and cₒ should produce similar or closely similar curves after adjustment.

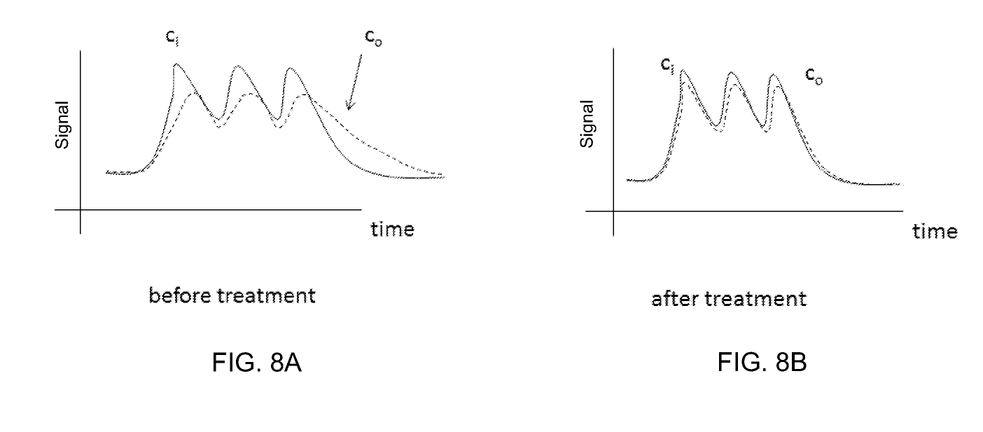

In the preferred embodiment, the same steps of determining concentration profiles are repeated during and/or after treatment, e.g., flow diverter placement. Repeating the steps of determining concentration profiles provide additional profiles cᵢ and cₒ demonstrating flow for during and/or after treatment. A comparison of these curves or profiles before and during and/or after treatment allows a quantitative assessment of the effectiveness of the treatment, e.g., flow diversion. FIG. 8A, shows an exemplary comparison of the proximal and distal profiles, cᵢ (solid line) and cₒ (dashed line), respectively, before treatment. FIG. 8B shows an exemplary comparison of the proximal and distal profiles, cᵢ (solid line) and cₒ (dashed line), after treatment. A difference between cᵢ and cₒ shows the wash-in and wash-out of contrast medium in the aneurysm. Referring to FIG. 8A, when cᵢ is greater than cₒ, that is the diastole peaks of cᵢ are higher than the diastole peaks of cₒ, then the contrast medium is being stored in the aneurysm. When cᵢ is less than cₒ, contrast medium is being washed away from the aneurysm. FIG. 8B shows an ideal situation when there is no or little difference between cᵢ and cₒ. That is, when the diastole and systole peaks of cᵢ and cₒ match or closely match one another, contrast medium is not diverted into the aneurysm, which mirrors the condition of the absence of aneurysm, as shown by FIG. 4B or FIG. 6B.

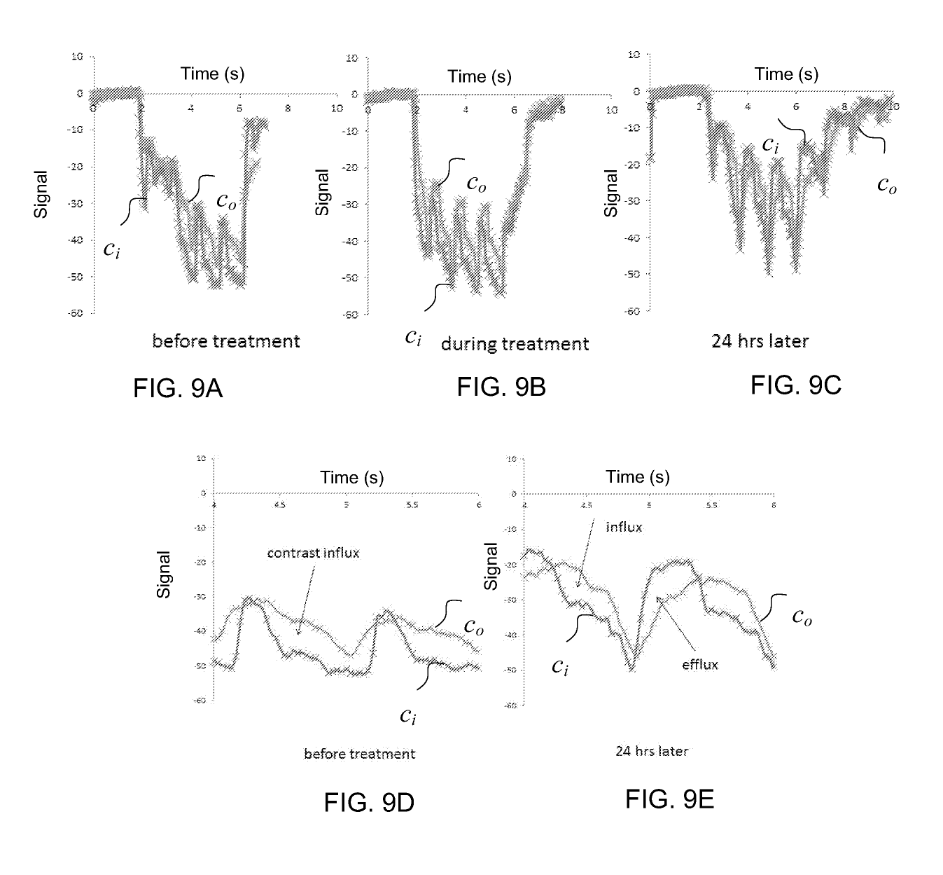

The following examples demonstrate the utility of the embodiments of the present disclosure. Reference is made to FIGS. 9A-9E. An aneurysm treated by flow diverters were imaged by high frame rate X-ray images before, right after and 24 hours after the treatment, shown in FIGS. 9A-9C, respectively. There is a considerable influx of contrast medium into the aneurysm before and during treatment in each cycle, so contrast medium was accumulated inside the aneurysm after each injection. FIGS. 9D and 9E show the portion of the concentration profiles of FIGS. 9A and 9C between 4-6 seconds, respectively. Areas of contrast influx and efflux can also be seen in FIGS. 9D and 9E. As shown, contrast accumulation before treatment is a result of strong intra-aneurysmal flow, but is caused by repeated injections and low flow diversion after treatment. Contrast transport is more balanced 24 hours after treatment. Contrast wash-in occurs at the acceleration phase of systole, and washout for the rest of the cycle. As a result, the accumulation of contrast in each cycle is much smaller, as shown in FIG. 9E. Another observation is that the time delay decreases after treatment, meaning that blood travels with little or no delay across the aneurysm neck after flow diversion.

The aneurysm in FIGS. 9A-9E is complex, consisting of multiple aneurysms. Nevertheless, the method of the present invention provides marked improvement in flow to a more normal flow. In keeping with the convention used herein, Cᵢ represents the flow at the segment proximal to the aneurysm and Cₒ represents the flow at the segment distal to it. In the plot labeled “before treatment” it can be seen in the curves do not have optimal overlap, indicating the difference in contrast agent concentration (the curves are corrected for time delay). The behavior improves marginally during treatment, and more improvement can be seen 24 hours after treatment. As also can be seen, wash-in and wash-out (accumulation and discharge of contrast agent within the aneurysm) improves at 24 hours as compared to before treatment.

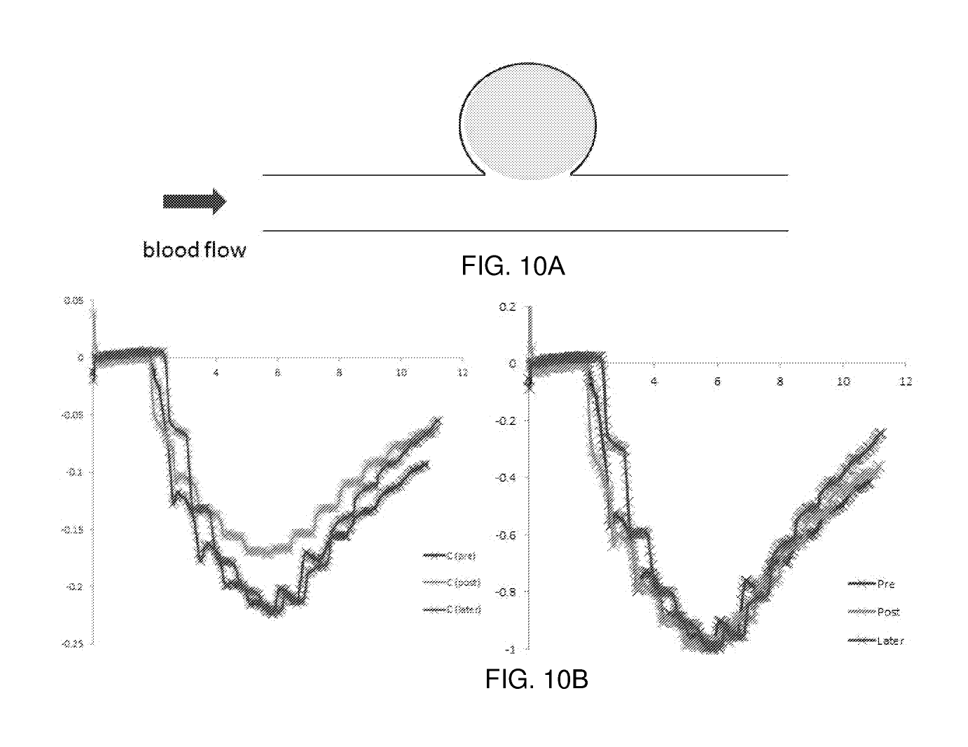

FIG. 10b schematically demonstrates the prior art method of evaluating flow diverters, which consists of measuring contrast agent behavior directly at the region of interest as opposed to proximal and distal locations. As can be seen, very little information can be discerned regarding the effectiveness of the flow diverter, as the before and after plots are largely superimposed.

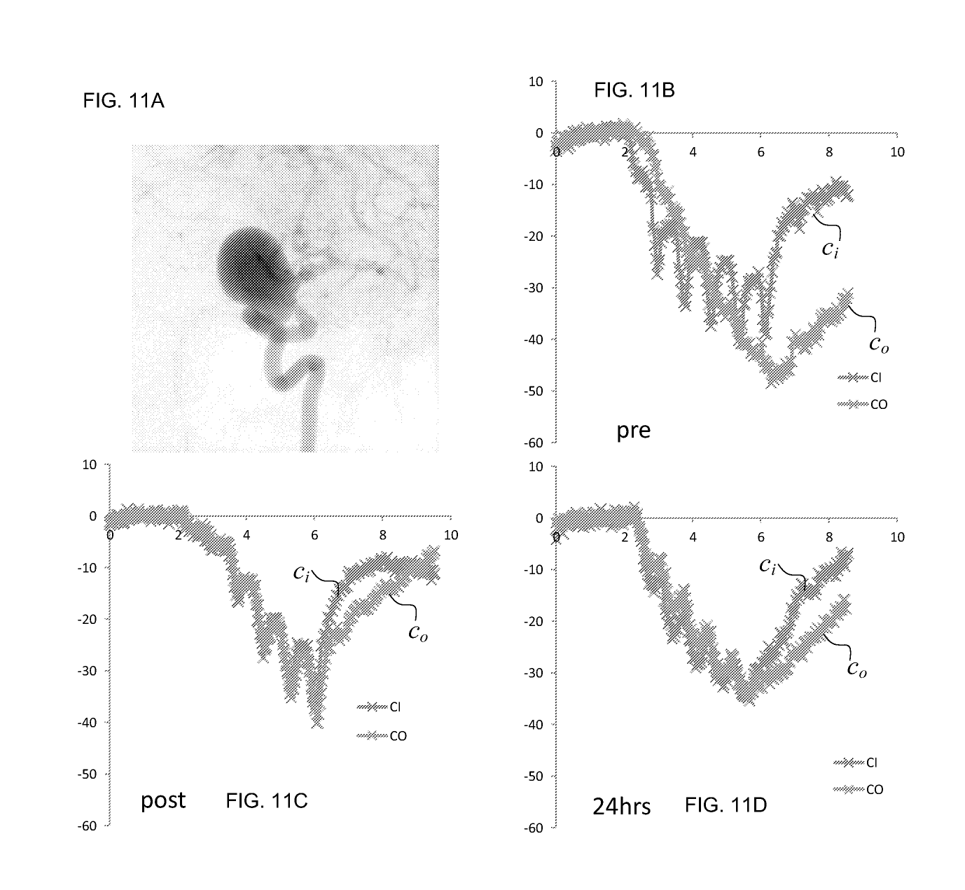

FIGS. 11B-11D demonstrates data for an aneurysm having some degree of vessel overlap, shown in FIG. 11A, which would complicate evaluation of diverter performance with the methods of the prior art. Because some vessel structure lies directly adjacent to the aneurysm site, visualization of contrast agent directly at the aneurysm is complicated. Nevertheless as seen graphically, the present method provides an immediate improvement of flow directly after introduction of a flow diverter (FIG. 11C) relative to the flow before the introduction of a flow diverter (FIG. 11B); and the improvement gets better at 24 hours after introduction of the diverter (FIG. 11D). The degree of contrast influx (into the aneurysm) and efflux (out of the aneurysm) decreases, indicating less flow diversion due to the aneurysm.

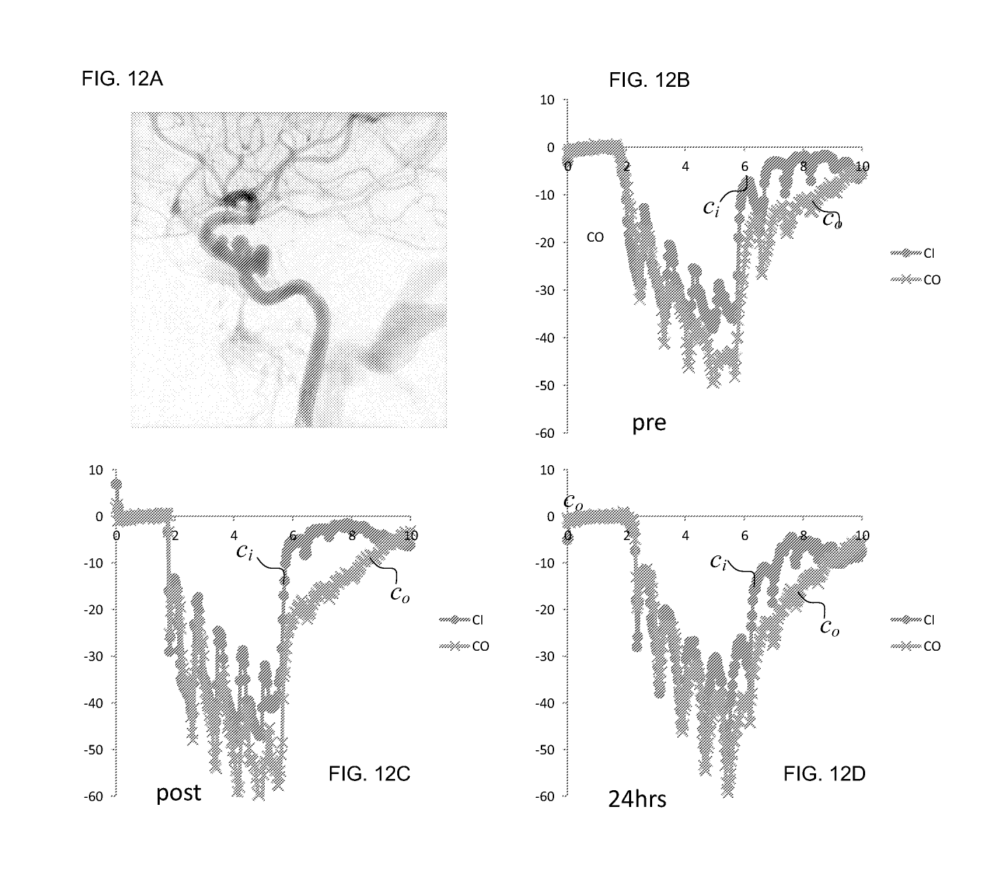

FIGS. 12B-12D demonstrates data for a complex aneurysm, which consists of four separate aneurysms, as shown in FIG. 12A. Comparing the concentration profiles obtained prior to treatment (FIG. 12B), immediately after treatment (FIG. 12C), and 24 hours after treatment (FIG. 12D), flow is markedly improved 24 hours after introduction of a flow diverter, as is evidenced by the improved overlap of the c, and cₒ curves.

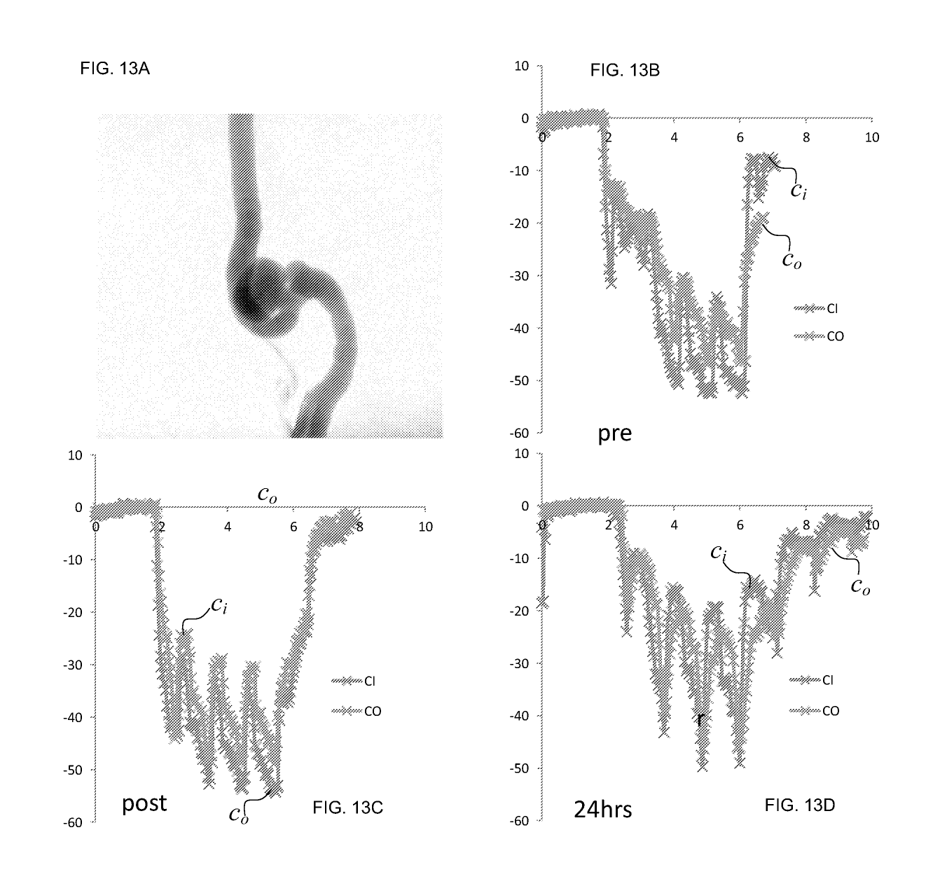

FIGS. 13B-13D provides data for a complex aneurysm, which consists of two separate aneurysms: one big downstream aneurysm and one smaller upstream aneurysm, as shown in FIG. 13A. In this case the proximal and distal segments were upstream and downstream, respectively, of the aggregate region of interest. Again, Comparing the concentration profiles obtained prior to treatment (FIG. 13B), immediately after treatment (FIG. 13C), and 24 hours after treatment (FIG. 13D), flow is markedly improved 24 hours after introduction of a flow diverter, as is evidenced by the improved overlap of the cᵢ and cₒ concentration curves. In this case, the traditional method will give inadequate information. Because the artery is looped around itself, measurement of contrast agent concentration at the region of interest will be problematic.

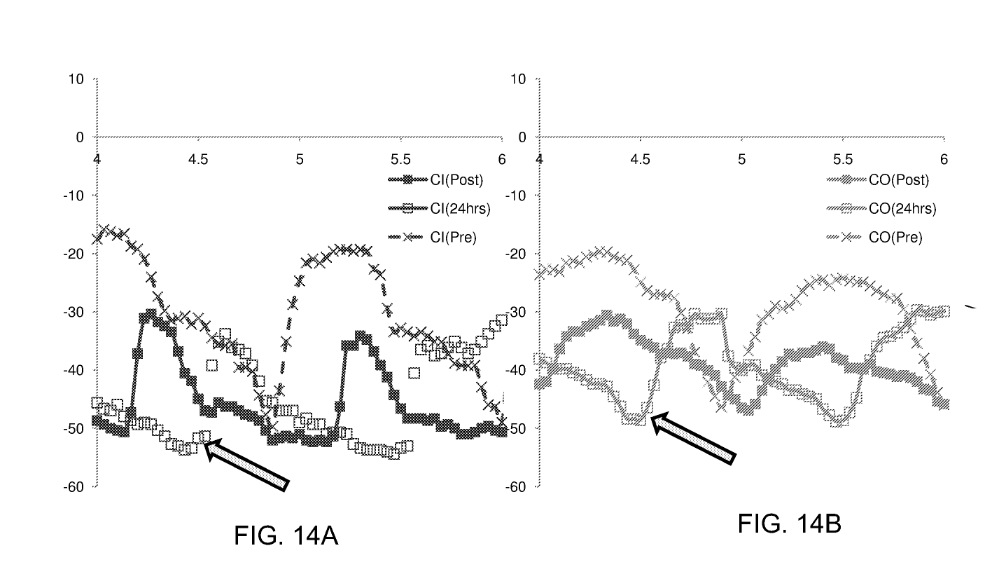

FIG. 14A shows the concentration profiles in FIGS. 13B-D of the proximal region of interest before treatment, immediately after treatment, and 24 hours later in the same graph. FIG. 14B shows the concentration profiles in FIGS. 13B-D of the distal region of interest before treatment, immediately after treatment, and 24 hours later in the same graph. FIGS. 14A-14B show how the flow behavior improves with time at 24 hours. The 24 hour curves for both cᵢ and cₒ, indicated by arrows in FIGS. 14A and 14B, resemble one another more closely than even the corresponding “post” curves. Although not intending to be bound by theory, this is likely indicative of thrombosis setting in over time in the aneurysm after introduction of the flow diverter from decreased influx into the aneurysm and resulting stagnation.

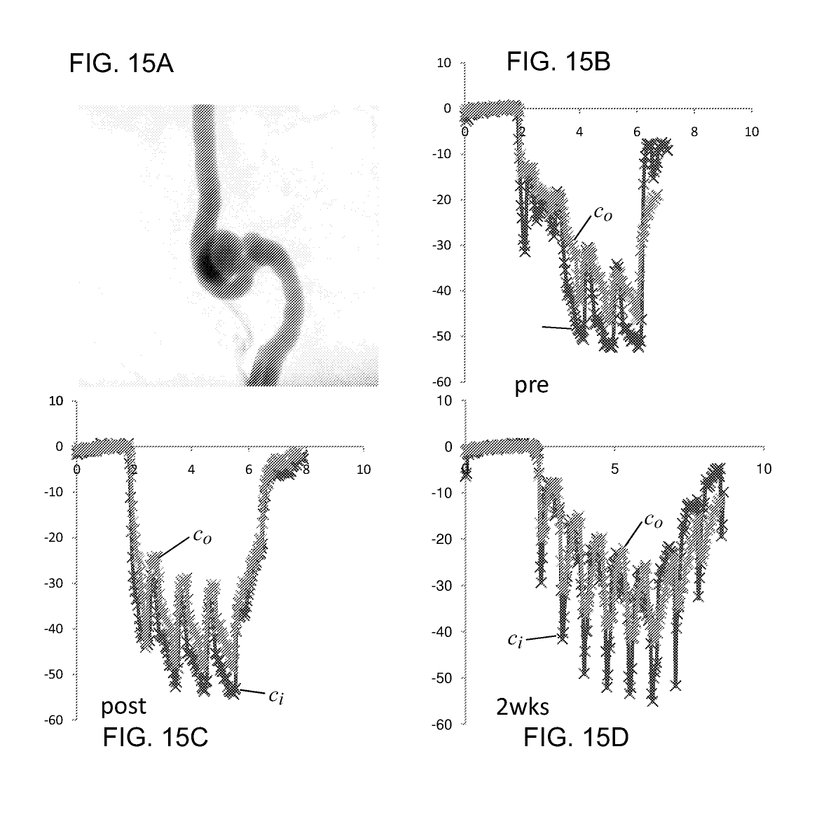

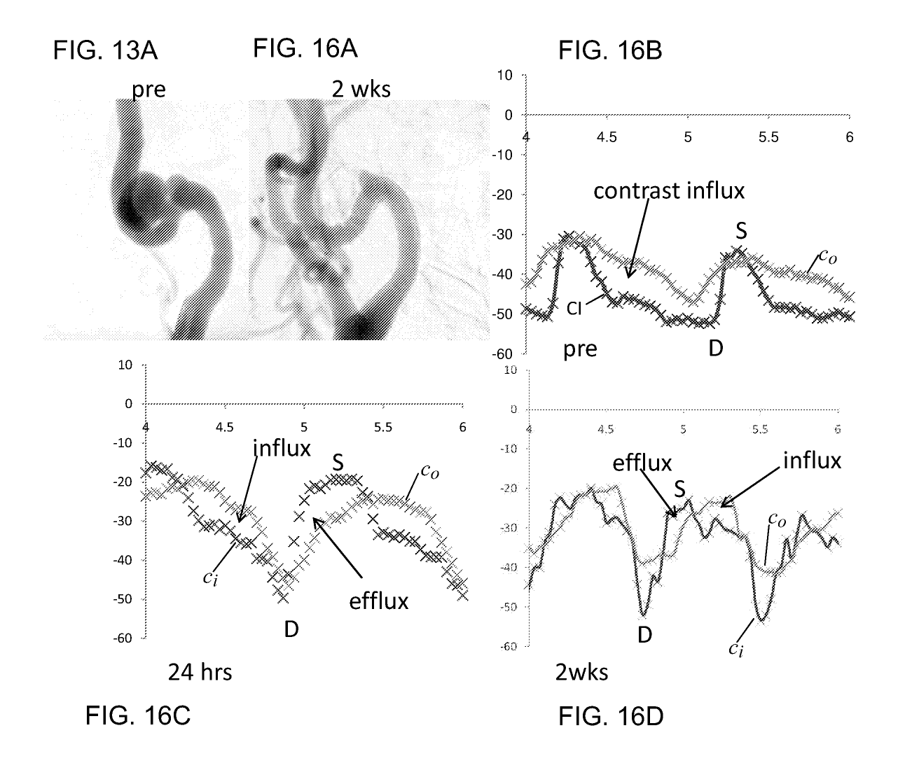

FIG. 15 shows the same system studied and presented in FIGS. 13A-D, with added data for the period of 2 weeks after introduction of a flow diverter. The improvement is more visible after a 2 week period. FIG. 16A is a photograph of the aneurysm of FIG. 13 taken 2 weeks after treatment. FIGS. 16B-D show the portion of the concentration profiles of FIGS. 13B, 13D and 15 between 4-6 seconds, respectively. A comparison of FIGS. 13B-D shows that the aneurysm has improved in appearance at 2 weeks after introduction of a flow diverter in comparison to a time prior to introduction. The improvement in flow behavior before introduction, 24 hours after introduction of the flow diverter, and 2 weeks after introduction of the flow diverter is evident from an examination of the influx and efflux behavior, as much less blood is flowing into and out of the aneurysm as time progresses.

### Example 2

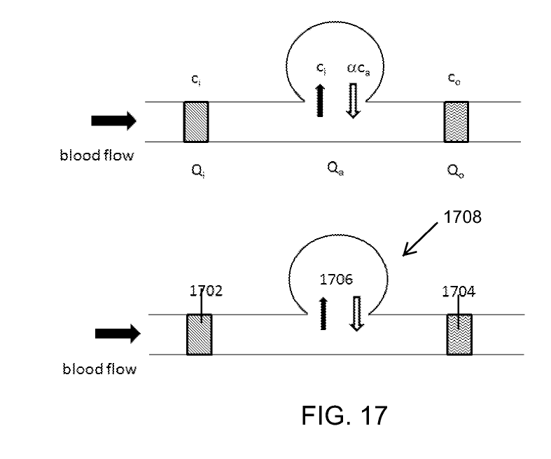

According to another aspect of the present disclosure, concentration profiles of the proximal and distal locations to an area of interest are determined using 3D DSA and a 2D DSA. Referring to FIG. 17, in the preferred embodiment, two regions of interest (ROI) 1702 and 1704 of blood vessel 1708, such as an internal carotid artery, experiencing abnormal flow were selected on the 2D DSA. ROI 1702 is proximal to aneurysm 1706, and ROI 1704 is distal to aneurysm 1706. ROI 1702 and ROI 1704 are preferably rectangular in shape and included sections of blood vessel 1708. Each ROI from the 2D DSA was projected back onto the 3D DSA after co-registration to determine the volume of the respective ROI. The volume was then used to normalize the total signal at each ROI. Another ROI, ROI 1710, that included the entire volume of aneurysm 1706 was selected, and the aneurysm signal was normalized by the aneurysm volume selected.

In the preferred embodiment, after the normalization, each ROI is represented by

$$c = \frac{1}{{V}_{ROI}} {\sum }_{ROI}^{ } ln ( \frac{I}{{I}_{m}} )$$

where I is the signal and Iₘ is the mask image signal. V_{ROI} is the volume associated with the respective enclosed ROI on the 2D DSA. In one embodiment, the signal I is lower than the mask image signal. As such, c is negative, but its magnitude is proportional to the local attenuation that was a function of contrast concentration. In another embodiment, the signal intensity can be converted into Hounsfield unit since c has a dimension of 1/meter.

Referring to FIG. 17, Q, represents the flow rate at proximal ROI 1702 and cᵢ represents the concentration of contrast medium of proximal ROI 1702. Qₒ represents the flow rate at distal ROI 1704 and cₒ represents the concentration of contrast medium at distal ROI 1704. Qₐ represents the flow rate at aneurysm ROI 1706 and cᵢ also represents the concentration of contrast medium flowing into aneurysm 1706 and αcₐ represents concentration of contrast medium flowing out of aneurysm 1706. Based on FIG. 17, the balance of contrast medium could be written as

Qᵢcᵢ−Qₐcᵢ+αQₐcₐ=Qᵢcₐ

where Q and c are the flow rate and concentration of contrast medium at the respective locations: the subscripts i, o, and a were for proximal ROI 1702, distal ROI 1704, and aneurysm 1706, respectively. Qᵢcᵢ is for the contrast medium coming into the system, and Qᵢcₒ the amount of contrast medium leaving the system. In the preferred embodiment, the amount of fluid entering aneurysm 1706 and leaving aneurysm 1706 is the same, which is the intra-aneurysmal flow Qₐ. Further, the concentration entering the aneurysm is cᵢ and the concentration leaving the aneurysm is proportional to cₐ, the aneurysmal concentration. This equation could be simplified as

(1−ƒ)cᵢ+afcₐ=cₒ

where

$$f = \frac{{Q}_{a}}{{Q}_{i}}$$

is the ratio of arterial flow rate that actually enters the aneurysm. Since cᵢ, cₐ, and cₒ are the temporal history of the concentration, they are preferably treated as vectors in the n-dimensional space, where n is the number of sampling points. In one embodiment, it is assumed that the base vectors in the null space of are vₖ (k=1, . . . , n−1). As such, the product of the previous equation can be summed with all the base vectors, resulting in the following equation:

$$( 1 - f ) {\sum }_{k}^{ } {v}_{k} ( {v}_{k} \cdot {c}_{i} ) = {\sum }_{k}^{ } {v}_{k} ( {v}_{k} \cdot {c}_{o} )$$

In one embodiment, (vₖ·cₐ)=0 by the definition of null space. With the time curves or concentration profiles (cᵢ and cₒ) measured from the respective ROIs, the intra-aneurysmal flow ratio ƒ is preferably determined by the following equation:

$$f = 1 - \frac{\vert {\sum }_{k}^{ } {v}_{k} ( {v}_{k} \cdot {c}_{o} ) \vert}{\vert {\sum }_{k}^{ } {v}_{k} ( {v}_{k} \cdot {c}_{i} ) \vert}$$

Based on this formula, the difference between cᵢ and cₒ determines the intra-aneurysmal flow rate. A close match between cᵢ and cₒ gives a low intra-aneurysmal flow. The use of cₐ is to find the base vectors of the null space, unlike in previous studies where all the aneurysmal dynamics depend only on the behavior of cₐ.

The following examples demonstrate the utility of the embodiments of the present disclosure. Six patients with seven internal carotid artery (ICA) aneurysms were studied. These aneurysms ranged from 7 mm to 31 mm, and the average size was 17 mm. Each patient received a 3D DSA and a 2D DSA on Siemens Axiom ArtisdBA (Siemens Medical Solutions, Germany) as part of the clinical protocol. In 2D DSA, non-diluted contrast medium (OMNIPAQUE 300; Amersham Health) was injected into the ICA at 2 mL/s for 4 seconds and the X-ray images were acquired at 30 frames/s. For the 2D DSA, each image was subtracted logarithmically by the mask image. In the preferred embodiment, a 3D DSA is acquired prior to the 2D DSA, and the 3D DSA permitted the planning of an optimal projection view for the subsequent 2D DSA. In another embodiment, the 3D DSA also provides size measurements of the parent artery and aneurysm that can be later used for signal processing. A high frame rate (30 f/s) provides a better estimate on intra-aneurysmal flow than low frame rates (7.5 f/s and 15 f/s). That is, it is preferred that a frame rate of higher than 7.5 f/s is used, it is more preferred that a frame rate of higher than 15 f/s is used, and it is most preferred that a frame rate of 30 f/s or higher is used.

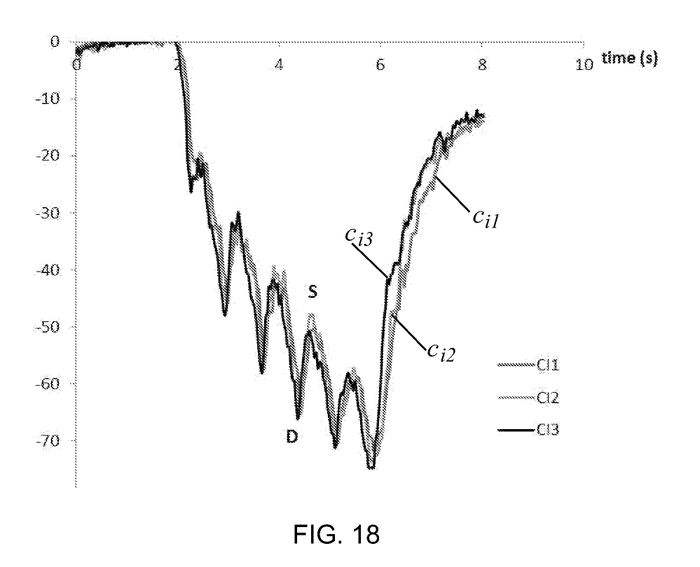

In the preferred embodiment, the proximal and distal regions of interest are selected within about 4 cm of the area of interest, e.g., aneurysm, and more preferably within about 2 cm. FIG. 18 shows the time-curves of the signal for three ROIs along the parent artery in one patient that are 2 cm apart. Because of the 2 s injection delay, no signal was seen in the first 2 s, and the contrast injection lasts for another 4 s before it decreases to the base line. As can be seen in FIG. 18, quantitative behavior does not change with the selection of different ROI along the parent vessel. This preferably ensures that the same dynamic feature is maintained when the ROI is selected within 4 cm of the aneurysm and the contrast distribution is not distorted by dispersion or diffusion. In FIG. 18, D denotes a diastole peak, and S indicates a systole peak. At systole, a higher blood flow rate dilutes the contrast injection immediately so a lower signal is seen at the peak systole. Similarly, contrast concentration is less diluted during diastole and gives a greater signal at diastole. Nevertheless, the magnitude of c represents the contrast concentration despite of the difference in the signal.

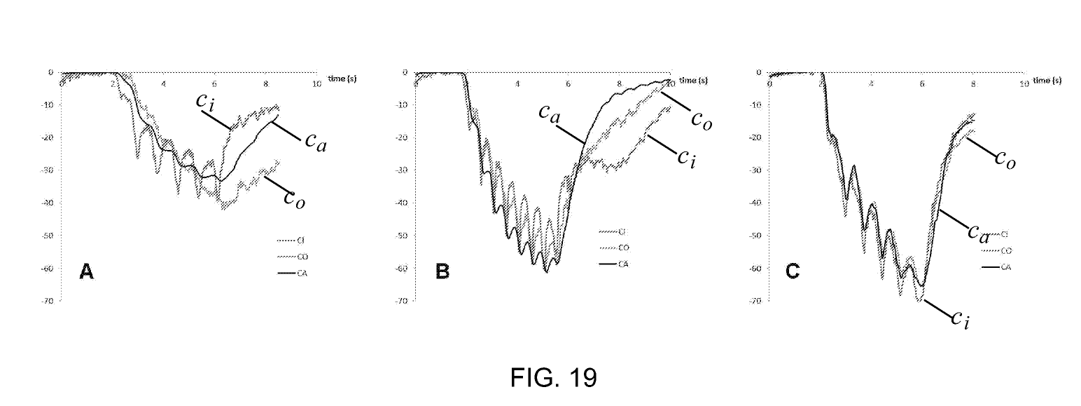

Dynamic behaviors of cᵢ, cₐ, and cₒ reveal the intra-aneurysmal hemodynamics in FIGS. 19A-C. These three cases with different ƒ are selected for a demonstration purpose. The flow ratio ƒ of FIG. 19A is 71%, of FIG. 19B is 56%, and of FIG. 19C is 19%. An aneurysm of high ƒ produces a cₒ curve that is very different from cᵢ or cₐ, and all the curves (cᵢ, cₐ, and cₒ) are similar for an aneurysm with small ƒ. A high ƒ implies a greater intra-aneurysmal flow and a rapid mixing of the contrast with blood, as shown by FIG. 19A, and the contrast concentration in the aneurysm is often less pulsatile. As a result, the distal contrast concentration loses the signal pulsatility. The contrast dynamic behavior for an aneurysm of low ƒ is dominated by the pulsatility, and pulsatility can be seen in all three concentration curves for all three cases.

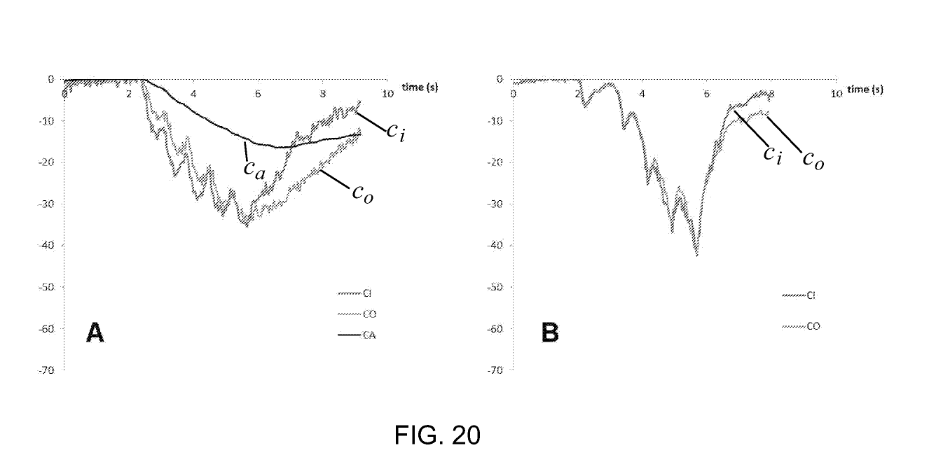

Treatment certainly alters the intra-aneurysmal hemodynamics and contrast dynamic behavior. FIG. 20A shows the dynamic behavior for the aneurysm after three flow diverters. The cₒ curve matches the cᵢ curve much more closely than the cₐ curve, indicating less flow into the aneurysm and more flow bypassing the aneurysm. The estimate of ƒ based on cerebral angiography decreases to nearly 0% from 71% before the treatment, implying a good flow diversion. FIG. 20B presents the ci- and co-curves after coil embolization. Because of the presence of coils, the ca-curve is not available; nevertheless, agreement between ci- and co-curve is improved, as compared to FIG. 19C. An estimate of ƒ shows that ƒ˜0% by assuming a degenerate space for the Cₐ. This figure not only shows that the difference due to the treatment is significant, but also demonstrates the possibility that these two curves (cᵢ and cₒ) can be used for evaluation of the hemodynamics in intracranial aneurysms in general.

According to one aspect of the present disclosure, it is preferred to normalize the signal intensity with the 3D DSA. Normalizing the signal by the area of an ROI may be convenient, but it does not account for the three-dimensionality of a complex arterial or aneurysmal geometry. When the vessel is not parallel to the projection plane where the 2D DSA is acquired, comparison between signals at various regions may be a challenge. For example, both a sphere and a cylinder with the same concentration of contrast medium appear on an angiogram as a circle, and normalizing the signal by the area of the circle does not produce a variable that is proportional to the contrast concentration for the sphere. Since 3D DSA provides visualization of anatomy and morphology for treatment planning and is already part of our existing clinical protocol, these 3D images offer us an additional opportunity for better quantifying the signal on 2D DSA.

A slow injection rate is required to produce signal pulsatility during the injection. In our cases, 2 ml/s is equivalent to half of the mean flow rate at the ICA, so the mean contrast concentration is approximately 33%. At this concentration, the signal can still be treated as linear to the concentration. Signal variation is much smaller once the concentration reaches 80% because the signal is an exponential function of the attenuation. Dynamic range of digital images limits the range of concentration that we can utilize. In certain embodiments involving aneurysms at the posterior cerebral circulation, this injection rate may need to be reduced accordingly.

According to another aspect, the embodiments of the present disclosure can be used to estimate the flow rate by knowing the linear attenuation coefficient for the contrast agent. For instance, the Hounsfiled unit for Omnipaque is 7300 at 125 kV and the linear attenuation coefficient of water is 16 m⁻¹, so the linear attenuation of Omnipaque is 133 m⁻¹. A 2 mL/s blood flow will give a 66 m⁻¹ attenuation because of 1-to-1 mixing of contrast injection and blood. The last few pulses before the end of contrast injection in FIG. 20 show the attenuation at diastole are 40 m⁻¹ for FIG. 19A, which corresponds to a diastolic flow rate of 2*133/40−2=4.65 mL/s. The diastolic flow rates for FIGS. 19B and 19C are close to 2 mL/s.

The embodiments of the systems of the present invention may include one or more computer systems to implement the various methods of the present invention. One exemplary computer system may include a central processing unit (CPU), which may be any general-purpose CPU. The present invention is not restricted by the architecture of the CPU or other components of the systems of the present invention as long as the CPU and other components support the inventive operations as described herein. The CPU may execute the various logical instructions according to embodiments of the present invention. For example, the CPU may execute the calculation of the concentration profiles according to the exemplary operational flows described above.

In addition, the exemplary computer system may also include random access memory (RAM), which may be SRAM, DRAM, SDRAM, or the like. The embodiments may also include read-only memory (ROM) which may be PROM, EPROM, EEPROM, or the like. The RAM and ROM hold user and system data and programs, as is well known in the art.

The exemplary computer system also includes input/output (I/O) adapter, communications adapter, user interface adapter, and display adapter. I/O adapter, user interface adapter, and/or communications adapter may, in certain embodiments, enable a user to interact with the computer system in order to input information and obtain output information that has been processed by the computer system.

The I/O adapter preferably connects to one or more storage device(s), such as one or more of hard drive, compact disc (CD) drive, floppy disk drive, tape drive, etc. to the exemplary computer system. The storage devices may be utilized when the RAM is insufficient for the memory requirements associated with storing data for operations of the elements described above (e.g., clam adjudication system, etc.). The communications adapter is preferably adapted to couple the computer system to a network, which may enable information to be input to and/or output from the computer system via the network (e.g., the Internet or other wide-area network, a local-area network, a public or private switched telephony network, a wireless network, any combination of the foregoing). The user interface adapter couples user input devices, such as keyboard, pointing device, and microphone and/or output devices, such as speaker(s) to the exemplary computer system. The display adapter is driven by the CPU to control the display on the display device, for example, to display the concentration profiles described above.

It shall be appreciated that the present invention is not limited to the architecture of the exemplary computer system. For example, any suitable processor-based device may be utilized for implementing the various elements described above (e.g., software for presenting the user interfaces, claim adjudication system, etc.), including without limitation personal computers, laptop computers, computer workstations, and multi-processor servers. Moreover, embodiments of the present invention may be implemented on application specific integrated circuits (ASICs) or very large scale integrated (VLSI) circuits. In fact, persons of ordinary skill in the art may utilize any number of suitable structures capable of executing logical operations according to the embodiments of the present invention.

Although the present invention and its advantages have been described in detail, it should be understood that various changes, substitutions and alterations can be made herein without departing from the spirit and scope of the invention as defined by the appended claims. The examples given are merely illustrative and not exhaustive. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the invention is intended to encompass within its scope such processes, machines, manufacture, compositions of matter, means, methods, or steps.

What is claimed is:

1. A method for evaluating hemodynamics of a region of interest, said method comprising the steps of:
injecting a contrast agent upstream of a region of interest of a blood vessel;
creating a plurality of 3D digital subtraction images and a plurality of 2D images of said blood vessel based at least on a measured signal of said injected contrast agent;
creating a plurality of digital division angiogram images from said 2D images;
selecting a first segment in said vessel at or near the proximal end of said region of interest, said first segment having a geometry characterized by having at least one axis being orthogonal to the centerline of the blood vessel;
measuring mean signal intensity for the first segment from said digital division angiogram images;
mapping said plurality of digital division angiogram images back to said 3D images to obtain a concentration profile of said first segment.

2. The method of claim 1 further comprising the steps of:
selecting a second segment in said blood vessel at or near the distal end of said region of interest, said second segment having a geometry characterized by having at least one axis being orthogonal to the centerline of the blood vessel;
measuring mean signal intensity for the second segment from said digital division angiogram images; and
mapping said plurality of digital division angiogram images back to said 3D images to obtain a concentration profile of said second segment.

3. The method of claim 2 further comprising the step of comparing the concentration profile of said first segment with the concentration profile of said second segment to determine the hemodynamics of the region of interest.

4. The method of claim 2 wherein said contrast agent is injected prior to treatment of said region of interest, wherein said treatment is configured to alter the hemodynamics of said region of interest.

5. The method of claim 4 further comprising the steps of:
injecting a contrast agent upstream of a region of interest of a blood vessel after said treatment;
creating a plurality of 3D digital subtraction images and 2D images of said blood vessel based on said injected contrast agent;
creating a plurality of digital division angiogram images from said 2D images;
measuring mean signal intensity for the first segment from said digital division angiogram images;
mapping said plurality of digital division angiogram images back to said 3D images to obtain a concentration profile of said first segment after said treatment;
measuring mean signal intensity for the second segment from said digital division angiogram images;
mapping said plurality of digital division angiogram images back to said 3D images to obtain a concentration profile of said second segment after said treatment; and
comparing the concentration profiles of the first and second segments obtained prior to treatment with the concentration profiles of the first and second segments obtained after said treatment to evaluate the performance of said treatment.

6. The method of claim 4 further comprising the steps of:
injecting a contrast agent upstream of a region of interest of a blood vessel during said treatment;
creating a plurality of 3D digital subtraction images and 2D images of said blood vessel based on said injected contrast agent;
creating a plurality of digital division angiogram images from said 2D images;
measuring mean signal intensity for the first segment from said digital division angiogram images;
mapping said plurality of digital division angiogram images back to said 3D images to obtain a concentration profile of said first segment after said treatment;
measuring mean signal intensity for the second segment from said digital division angiogram images;
mapping said plurality of digital division angiogram images back to said 3D images to obtain a concentration profile of said second segment after said treatment; and
comparing the concentration profiles of the first and second segments obtained prior to treatment with the concentration profiles of the first and second segments obtained during said treatment to determine whether additional treatment is needed.

7. The method of claim 4 wherein said treatment comprises use of flow diverter.

8. The method of claim 4 wherein said treatment comprises coil embolization.

9. The method of claim 1, wherein the region of interest is an aneurysm.

10. The method of claim 1, wherein the region of interest is the site of atherosclerotic disease.

11. The method of claim 1, wherein said 3D images and said 2D images are X-ray images.

12. The method of claim 1, wherein said 2D images are obtained at a frame rate of 30 f/s or greater.

13. The method of claim 1 wherein said first segment and said second segment are substantially equidistant from said region of interest.

14. The method of claim 1, wherein said contrast agent comprises (1-N,3-N-bis(2,3-dihydroxypropyl)-5-[N-(2,3-dihydroxypropyl)acetamido]-2,4,6-triiodobenzene-1,3dicarboxamide).

15. The method of claim 1, wherein said step of injecting a contrast agent comprises injecting a contrast agent at an injection rate of between about 1 mL/s and 3 mL/s.

16. The method of claim 1, wherein said step of injecting a contrast agent comprises injecting a contrast agent at an injection rate of less than 2 mL/s.

17. A method for evaluating hemodynamics of a region of interest, said method comprising the steps of:
injecting a contrast agent upstream of a region of interest of a blood vessel;
creating a plurality of 3D digital division angiogram images and a plurality of 2D digital division angiogram images of said blood vessel based at least on a measured signal of said injected contrast agent;
selecting a first segment in said vessel at or near the proximal end of said region of interest, said first segment having a geometry characterized by having at least one axis being orthogonal to the centerline of the blood vessel;
determining the volume of said first segment, said determining comprises projecting said plurality of said 2D digital subtraction angiogram images back to said 3D digital subtraction angiogram images;
obtaining a concentration profile for said first segment using said determined volume to normalize the total signal of the injected contrast agent at said first segment.

18. The method of claim 17 further comprising the steps of:
selecting a second segment in said vessel at or near the proximal end of said region of interest, said first segment having a geometry characterized by having at least one axis being orthogonal to the centerline of the blood vessel;
determining the volume of said second segment, said determining comprises projecting said plurality of said 2D digital subtraction angiogram images of said second segment back to said 3D digital subtraction angiogram images; and
obtaining a concentration profile for said second segment using said determined volume to normalize the total signal of the injected contrast agent at said second segment.

19. The method of claim 18 further comprising the step of comparing the concentration profile of said first segment with the concentration profile of said second segment to determine the hemodynamics of the region of interest.

20. The method of claim 19 wherein said comparing step comprises determining a flow rate in the region of interest based at least on the difference between the concentration profile of the first segment and the concentration profile of the second segment.
